(19) United States
(12) Patent Application Publication
(10) Pub. No.: US 2023/0218386 A1
(43) Pub. Date: Jul. 13, 2023

(54) Ligament Supplementation

(71) Applicant: Robby Turk, Charlotte, NC (US)

(72) Inventor: Robby Turk, Charlotte, NC (US)

(21) Appl. No.: 17/647,456

(22) Filed: Jan. 8, 2022

(51) Int. Cl. A61F 2/08; A61B 17/04

(52) U.S. Cl. CPC A61F 2/08; A61B 17/0401; A61F 2002/0882; A61B 2017/0414

(57) ABSTRACT

Ligament injury is a common injury among both high-level and everyday athletes. There is increasing research demonstrating the factors that place individuals at increased risk of ligament injury. These include, biological, genetic, morphological, anatomic, neuromuscular, hormonal, gender, activity-related, environmental, and psychological factors among others. The present invention presents a novel native ligament-graft complex and method by which a native ligamentous or tendinous structure in the human or animal body may be supplemented in order to decrease risk of future injury. This invention involves supplementation of the native ligament in human or animal subjects through the use of autograft, allograft, biologic, and/or synthetic graft incorporation and fixation to native tissue for the purpose of increasing the threshold to injury for the native ligament.

### FIELD OF INVENTION

The present invention relates to a method, apparatus, and system by which a native ligament is supplemented.

### BACKGROUND/PROBLEM

A ligament is a fibrous band of connective tissue that connects one bone to another and provides stability at joints such as, but not limited to, the shoulder, elbow, knee, and ankle. These ligaments are prone to injury when excessive force is placed on the ligament, especially in the setting of sports or other athletic participation. One of the most well-known of these ligaments is the anterior cruciate ligament (ACL) in the knee, which is responsible for preventing excessive translation and rotation of the tibia (lower leg) relative to the femur (upper leg). Over 150,000 ACL injuries occur in the United States every year. This presents a significant loss in quality of life for patients. For high-level athletes, in particular, ACL injuries may potentially have a significant impact on their professional livelihood and future earning potential. The inconsistency in return to previous level of play and risks of additional injuries currently inherent in the ACL recovery process with the standard of care ACL repair/reconstruction and rehabilitation, has initiated various attempts to decrease the risk of ACL injury through preventative techniques. These have included physical therapy, modified and targeted strength training, and external bracing, among others.

Even with these recent attempts to prophylactically lower rates of ligament injury, the rate of injuries to ligaments such as the ACL, ulnar collateral ligament (UCL) and others has continued to be a significant burden to athletes, sports organizations, and the healthcare system. Interventions such as specialized and targeted physical therapy to correct mechanical factors and techniques that increase risk of ligament rupture, especially in women and others at additional increased risk, are inconsistent and difficult to access and adequately accomplish for a majority of athletes who may have limited resources.

A significant issue that has limited willingness to prophylactically intervene to prevent these injuries is that the factors contributing to risk of ACL and/or other ligament rupture have not been fully understood, defined, or quantified. However, there is growing evidence that genetic, hormonal, structural, psychologic, and mechanical factors, among others, all contribute to the risk that a patient has of injuring their ACL, or other ligaments, in the future. The increase in capabilities to assess individual risk of ligament injury presents opportunity for more direct and enhanced intervention to prevent this costly injury. Surgical intervention to prevent injury in a patient with significantly or substantially elevated risk is already an accepted theory in orthopaedics. For example, pediatric patients with Slipped Capital Femoral Epiphysis (SCFE) in one hip have an 18-73% elevated risk of also having subsequent SCFE in their other hip. In many of these cases, prophylactic fixation of the unaffected, contralateral hip at increased risk of injury is commonly performed and regarded as beneficial. However, the use of surgical and other types of intervention to prevent native ligament rupture based on elevated risk of injury has yet to be properly described and utilized in the art.

### SUMMARY/SOLUTION

The present invention meets the unmet needs of the art, as well as others, by describing an apparatus, method, and system by which a native ligament or tendon is supplemented to strengthen and/or increase stability of said ligament or tendon in order to prophylactically decrease the risk of injury to said ligament or tendon in the future.

Elements:

The invention includes, but is not limited to, the following elements:

Identification of elevated risk for ligament rupture secondary to clinical, radiographic, biologic, physiologic, hormonal, genetic, chemical, mechanical, and/or activity related factors in a human, animal, or non-biological simulation subject.

Intervention to structurally or biologically support or reinforce the native ligament with the goal of decreasing the likelihood of first-time injury to said ligament in the future.

Use of a tendon or ligament allograft for the supplementation, reinforcement, and/or support of the native ligament.

Creation of a graft-native ligament complex whose purpose may be to increase the capacity for said complex to experience various levels of force/stress without injury to the native ligament, graft-native-ligament complex, and/or surrounding structures.

Fixation of the graft-native ligament complex to the native ligament footprint using one or more suture anchors, for example 2-4 suture anchors on each of the proximal and distal native ligament attachment sites, respectfully.

The invention may also consist of the following optional elements:

Quantifiable risk of ligament injury above a specific threshold or otherwise deemed to be substantial compared to the risks associated with the intervention that may be described by the method of this invention.

Use of and/or application of a biologic patch on or around the native ligament as part of native ligament support, strengthening, supplementation or improvement.

Injection or addition of biologic agent such as pluripotent stem cells, hyaluronic acid or other agent that may enhance the strength, resistance, and/or stability of the native ligament, the graft-native ligament complex, or other surrounding and/or supporting structures, and/or improve the processes of graft incorporation and/or ligamentization.

Use of microfracture, ligament or soft tissue puncture/scarring, mechanical stimulation, or any other method of tactical localized trauma or tissue manipulation to increase blood flow or stem cell presence at or around the sight of procedure.

The chosen graft may go through various sterilization processes such as high-dose or low-dose irradiation to minimize risk of infection and other graft-related complications.

Use of computer or robot-assisted planning and/or intervention for purposes of better assessing native joint biomechanics and/or optimizing graft placement.

Fixation of the graft-native ligament complex to the native ligament footprint using one or more bone tunnels with implant fixation within or external to said bone tunnel.

Use of a synthetic graft including but not limited to a suture, tape suture, scaffold, or 3D printed element.

Use of a tendon or ligament autograft for the supplementation, reinforcement, and/or support of the native ligament.

Fixation of graft solely to native ligament without attachment to native ligament attachment sites.

Interrelationships:

The intervention is pursued as a result of a calculated or general radiographical, biologic, genetic, hormonal, psychologic, morphologic, biomechanical, and/or clinical judgement of elevated risk of ligament injury.

A graft is introduced adjacent to or circumferentially around the native ligament to create a naïve graft-native ligament complex.

Suture from one or more suture anchor or cortical fixation devices is utilized to further connect and reinforce the naïve graft-native ligament complex, forming a mature graft-native ligament complex.

This mature graft-native ligament complex is then fixated to the bone utilizing suture anchors to attach said complex directly into or near the native ligament footprint both proximally and distally.

### EMBODIMENT

In the first embodiment, the present invention provides a method, apparatus, and system by which there is internal supplementation of a native ligament or tendon in a human or animal subject with an allograft to prevent future injury of said ligament or tendon utilizing an arthroscopic or minimally-invasive method of intervention. This may be performed by creating a graft-native ligament complex and fixating that complex to the native ligament attachment site with the use of suture anchors to attach this soft tissue complex to bone as shown in FIG. 2A, FIG. 2B, and FIG. 2C.

### (Alternative) Embodiments

Use of a biological adherent to fixate the graft to the native ligament and/or circumferentially around the native ligament footprint.

A method and apparatus by which there is internal supplementation of a native ligament or tendon in a human or animal subject to prevent future injury of said ligament or tendon utilizing an open surgical approach method of intervention.

A method and apparatus by which there is internal supplementation of a native ligament or tendon in a human or animal subject to prevent future injury of said ligament or tendon utilizing injection of a biologic agent.

A method and apparatus by which there is internal supplementation of a native ligament or tendon in a human or animal subject to prevent future injury of said ligament or tendon utilizing the addition of a biological or non-biological material, substance, or implant that enhances the force and/or stress capacity of the ligament when sprayed, coated, injected, or directly applied into or onto the native ligament.

A method and apparatus by which there is internal supplementation of a native ligament or tendon in a human or animal subject to prevent future injury of said ligament or tendon utilizing an intervention by robotic or otherwise technologically controlled and/or directed device that may or may not require human direction and/or control during the procedure.

A method and apparatus by which there is internal supplementation of a native ligament or tendon in a human or animal subject to prevent future injury of said ligament or tendon utilizing a synthetic graft with fixation to the native ligament of tendon footprint through use of suture anchors, bone tunnels, and/or cortical fixation.

### Operation—(Alternative Embodiments)

Operation utilized with alternative embodiments may be similar to the operation of the first embodiment, but also may vary in the following ways:

Use of looped, single-bundle, or multi-bundle autograft (including but not limited to components of the hamstring tendon, patellar tendon, quadriceps tendon, iliotibial band, tibialis anterior tendon, palmaris longus tendon, or any combination thereof), xenograft, 3-D printed, or other synthetic graft for native ligament supplementation to create the graft-native ligament complex.

The order of suture anchor fixation may begin with the distal ligament attachment rather than the proximal attachment site.

Use of cortical fixation device utilizing bone tunnel(s) and/or bone bridge(s) to allow fixation of graft-native ligament complex to bone as is shown in FIG. 3A, FIG. 3B, and FIG. 3C.

Use of bone tunnel(s) and/or bone bridges and cortical fixation using interference screw(s) to achieve fixation.

Use of various suture and surgical knot tying techniques to create graft-native ligament complex and attach said complex to bone and/or soft tissue of subject without the additional fixation of suture anchor(s) or cortical fixation device.

Use of various extra-articular reconstruction methods with the aim of supplementing the native, uninjured ligament with or without formation of the graft-native ligament complex.

The graft used to supplement the native ligament may be placed adjacent to the portion of the ligament exposed to the highest mechanical force when in positions that hold the ligament at highest risk of rupture as illustrated by the ACL graft supplementation being placed adjacent to the anteromedial bundle in FIG. 5A, FIG. 5B and FIG. 5C.

The graft used to supplement the native ligament may be placed and/or attached adjacent to the native ligament without regard to specific force vectors acting upon the native ligament or graft-native ligament complex.

The graft attachment to the native ligament to create the graft-native ligament complex may utilize a number of techniques intertwining the two such as using suture and arthroscopic knot tying, staples, biologic adherent, or a combination thereof.

An additional number of bone bridges or tunnels used for cortical fixation of the graft-tendon complex compared to the one proximal and one distal bone bridge/bone tunnel illustrated in the alternative method in FIG. 3A, FIG. 3B and FIG. 3C.

The use of one or more bone bridges or tunnels with interference screw(s), cortical button(s), suture, other forms of tissue fixation, or any combination thereof for proximal and/or distal attachment of the graft and/or its suture to the native ligament and/or bone.

The creation of a graft-native tendon complex without direct fixation of the graft to the ligament attachment site/footprint or bone.

The first method performed with computer assistance for planning, analysis, execution, and/or evaluation of effectiveness and/or effect of intervention.

The first method performed with robotic assistance for planning, analysis, execution, and/or evaluation of effectiveness and/or effect of intervention.

Additional puncture or targeted trauma of the surrounding bony infrastructure may be performed in order to promote healing and/or integration of the graft to the native ligament and footprint.

Micropuncture, scarring, and/or targeted trauma of the native ligament and or the area near its footprint may be performed in order to promote healing and/or integration of the graft to the native ligament and footprint.

### DETAILED DESCRIPTION

In the first embodiment, the allograft tendon graft (210) is manipulated to an appropriate length and thickness (212) to accommodate the native ligament and, through one or more arthroscopic entry portals, is wrapped circumferentially around the native ligament (102). The opposing ends of the graft are then sutured to one another to maintain circumferential coverage of the native ligament, forming a naïve version of a graft-native ligament complex (220). Additional suture attached to a suture anchor is then utilized to further bound and reinforce the graft-native ligament complex (206), creating a mature graft-native ligament complex. This mature graft-native ligament complex is then fixated into or near a proximal ligament attachment site, also known as the ligament footprint (202). This process is repeated for one or more additional proximal attachment site suture anchors. Similarly, through the same or other arthroscopic portals, one or more suture anchors are used to fixate the distal aspect of the graft-native ligament complex to the distal attachment site (204). In the first described method, this may include 2-4 suture anchors per attachment site. Consequently, the new graft-native tendon complex exhibits a larger diameter and is now likely a stronger construct at time-zero compared to the native ligament alone in all degrees of flexion and extension of the joint. If not stronger in all degrees of flexion and extension, it is expected, at minimum, to be stronger at the flexion and extension angles that put the native ligament at highest risk of injury. The strength of this complex may change after biological healing and ligamentization of the construct. It is also in anatomical position and able to undergo increased force and stress without injury to the native ligament, thus decreasing likelihood of future injury. An illustration of this method is illustrated using the anterior cruciate ligament in FIG. 2A, FIG. 2B, and FIG. 2C and the Ulnar Collateral Ligament in FIGS. 4A and 4B.

In an alternative embodiment related to FIG. 3A-C, the allograft tendon graft is manipulated to an appropriate length and thickness (212) to accommodate the native ligament and, through one or more arthroscopic entry portals, is wrapped circumferentially around the native ligament (102). The opposing ends of the graft are then sutured to one another to maintain circumferential coverage of the native ligament, forming a naïve version of a graft-native ligament complex (220). Additional suture is then utilized to further bound and reinforce the graft-native ligament complex (206), creating a mature graft-native ligament complex. A Kirschner or other form of guide wire is then used to drill a small hole along the presumed track of the femoral and tibial tunnels, respectively. These tunnels should approximate a similar entry angle as the native ligament footprint, similar to the well-documented technique used in ligament reconstruction for said ligament. An appropriately sized drill may then be used to drill a larger bone tunnel in both the femur (302) and tibia (304), respectively, which will be used to attach the graft-native ligament complex (220) to the cortical fixation device (306). Suture is used to attach the reinforced mature graft-native ligament complex and also passed through the cortical fixation device. A receiver is passed through the respective bone tunnel to fetch the cortical fixation device, which is then pulled through along with its attached suture until it has exited the bone tunnel. The suture attached to the cortical fixation device is then tensioned from within the joint to provide stable fixation. This process is repeated for the distal bone tunnel (304). Consequently, the new graft-native tendon complex exhibits a larger diameter and is now likely a stronger construct at time-zero compared to the native ligament alone in all degrees of flexion and extension of the joint. If not stronger in all degrees of flexion and extension, it is expected, at minimum, to be stronger at the flexion and extension angles that put the native ligament at highest risk of injury. The strength of this complex may change after biological healing and ligamentization of the construct. It is also in anatomical position and able to undergo increased force and stress without injury to the native ligament, thus decreasing likelihood of future injury. The use of bone tunnels and/or bridges in this method is not the first described method due to the increased healing time and additional morbidity to native tissue compared to the first described method, however it is a potential embodiment of the invention.

In an alternative embodiment related to FIG. 5A-C, the allograft tendon graft (210) is manipulated and/or bundled to an appropriate thickness to ensure a minimum resulting graft-native ligament complex diameter. Through one or more arthroscopic tunnels, the graft is then placed adjacent to the native ligament and bound to said ligament by suture or some other adherent, thus forming a mature graft-native ligament complex. In this embodiment, the graft is placed adjacent to the aspect of the ligament at high risk of injury. In illustration 5A-C, depicting this procedure with an anterior cruciate ligament, the aspect of the ligament at highest risk of injury is thought to be the anteromedial bundle, which studies indicate is put under the most stress at low angles of knee flexion, when the ACL is most likely to be injured. Suture anchors are then utilized to fixate this complex to or near the native ligament proximal and distal attachment sites (202 and 204, respectively). In the present embodiment, this may include one or more suture anchors per attachment site. Consequently, the new graft-native tendon complex exhibits a larger diameter and is now likely a stronger construct at time-zero compared to the native ligament alone in all degrees of flexion and extension of the joint, but most specifically at points of flexion and extension thought to be at highest risk of ligament injury. The strength of this complex may change after biological healing and ligamentization of the construct. It is also in an anatomical position potentially best capable of increasing capacity of ligament to undergo force and stress without injury to the native ligament, thus decreasing likelihood of future injury.

### DESCRIPTION OF DRAWINGS/ILLUSTRATIONS

For the purpose of simplification, the following illustrations of the method defined in the present invention use the anterior cruciate ligament and ulnar collateral ligament as an example of how the invention and potential embodiments may be employed. These should not be construed as limitations on the scope of the invention, but rather as an exemplification of one embodiment thereof. Many other variations are possible including the use of said method on the deltoid ligament of the ankle or the coracoclavicular ligament of the shoulder, to name a couple of the many examples. Accordingly, the scope of the invention should be determined not by the embodiments illustrated, but by the appended claims and their legal equivalents.

FIG. 1A is a front elevational view of a flexed right human knee joint having the skin and muscle tissue removed along with the patella, for ease of view of the intact anterior cruciate ligament.

FIG. 1B is a front elevational view of an extended right human knee joint having the skin and muscle tissue removed along with the patella, for ease of view of the intact anterior cruciate ligament.

FIG. 1C is a side view of a portion of a typical right knee joint, in the plane of the anterior cruciate ligament, partly in section with part of the lateral condyle of the femur removed along with all the external ligaments and the patella for ease of view of native knee joint. The tibia is slightly translated anteriorly to accentuate the space between the anterior and posterior cruciate ligament and their tibial attachment sites.

FIG. 2A is a front elevational view of a flexed right human knee joint having the skin and muscle tissue removed along with the patella, for ease of view of anterior cruciate ligament supplementation utilizing a split graft wrapped circumferentially around the native ligament and anchored by 2 suture anchors used to fixate the graft-tendon complex to the area near the base of the native ACL femoral and tibial footprints, respectfully, in accordance with a first embodiment of the present invention.

FIG. 2B is a front elevational view of an extended right human knee joint having the skin and muscle tissue removed along with the patella, for ease of view of anterior cruciate ligament supplementation utilizing a split graft wrapped circumferentially around the native ligament and anchored by 2 suture anchors used to fixate the graft-tendon complex to the area near the base of the native ACL femoral and tibial footprints, respectfully, in accordance with another embodiment of the present invention.

FIG. 2C is a side view of a portion of a typical right knee joint, in the plane of the anterior cruciate ligament, partly in section with part of the lateral condyle of the femur removed along with all the external ligaments and the patella for ease of view of anterior cruciate ligament supplementation utilizing a split graft wrapped circumferentially around the native ligament and anchored by 2 suture anchors used to fixate the graft-tendon complex to the area near the base of the native ACL femoral and tibial footprints, respectfully, in accordance with another embodiment of the present invention.

FIG. 2D is a frontal view of the first described method of graft-native ligament complex creation illustrating a tendon allograft of appropriate length that is split to accommodate the circumference of the native ligament and is wrapped circumferentially around said ligament. The opposing, unattached ends of the graft may then be attached to one another by surgical knot tying, staple, surgical glue or a myriad of other potential techniques to maintain the two adjacent ends of the graft as it is wrapped circumferentially around the native ligament. This graft-native tendon complex may then be further bundles by sutures before anchored into or near the native ligament footprint as illustrated in FIG. 2A.

IG. 3A is a front elevational view of a flexed right human knee joint having the skin and muscle tissue removed along with the patella, for ease of view of anterior cruciate ligament supplementation utilizing a split graft wrapped circumferentially around the native ligament creating a graft-native ligament complex. Said graft-native ligament complex is fixated to the subject through a femoral and tibial bone bridge that leads to a cortical fixation device on both the femur and tibia, respectfully, in accordance with one of the multiple alternative embodiments of the present invention.

FIG. 3B is a front elevational view of an extended right human knee joint having the skin and muscle tissue removed along with the patella, for ease of view of anterior cruciate ligament supplementation utilizing a split graft wrapped circumferentially around the native ligament creating a graft-native ligament complex. Said graft-native ligament complex is fixated to the subject through a femoral and tibial bone bridge that leads to a cortical fixation device on both the femur and tibia, respectfully, in accordance with one of the multiple alternative embodiments of the present invention.

FIG. 3C is a side view of a portion of a typical right knee joint, in the plane of the anterior cruciate ligament, partly in section with part of the lateral condyle of the femur removed along with all the external ligaments and the patella for ease of view of anterior cruciate ligament supplementation utilizing a split graft wrapped circumferentially around the native ligament creating a graft-native ligament complex. Said graft-native ligament complex is fixated to the subject through a femoral and tibial bone bridge that leads to a cortical fixation device on both the femur and tibia, respectfully, in accordance with one of the multiple alternative embodiments of the present invention.

FIG. 4A is a side view of a portion of a typical right elbow joint, in the plane of the ulnar collateral ligament with all the external ligaments, joint capsule, and musculature removed for ease of view of the ulnar collateral ligament consisting of an anterior, posterior and oblique band.

FIG. 4B is a side view of a portion of a typical right elbow joint, in the plane of the ulnar collateral ligament with all the external ligaments, joint capsule, and musculature removed for ease of view of the ulnar collateral ligament consisting of an anterior, posterior and oblique band for ease of view of supplementation of the anterior band utilizing a split graft wrapped circumferentially around the native ligament and anchored by 2 suture anchors used to fixate the graft-tendon complex to the area near the base of the native UCL ulnar and humeral footprints, respectfully, in accordance with an embodiment of the present invention.

FIG. 5A is a front elevational view of a flexed right human knee joint having the skin and muscle tissue removed along with the patella, for ease of view of anterior cruciate ligament supplementation utilizing a graft attached adjacent to the anteromedial bundle of the anterior cruciate ligament and anchored by 1 suture anchor used to fixate the graft-tendon complex to the area near the base of the native ACL anteromedial bundle footprint on both the femoral and tibial attachments in accordance with an alternative embodiment of the present invention.

FIG. 5B is a front elevational view of an extended right human knee joint having the skin and muscle tissue removed along with the patella, for ease of view of anterior cruciate ligament supplementation utilizing a split graft wrapped circumferentially around the native ligament and anchored by 2 suture anchors used to fixate the graft-tendon complex to the area near the base of the native ACL femoral and tibial footprints, respectfully, in accordance with an embodiment of the present invention.

FIG. 5C is a side view of a portion of a typical right knee joint, in the plane of the anterior cruciate ligament, partly in section with part of the lateral condyle of the femur removed along with all the external ligaments and the patella for ease of view of anterior cruciate ligament supplementation utilizing a split graft wrapped circumferentially around the native ligament and anchored by 2 suture anchors used to fixate the graft-tendon complex to the area near the base of the native ACL femoral and tibial footprints, respectfully, in accordance with an embodiment of the present invention.

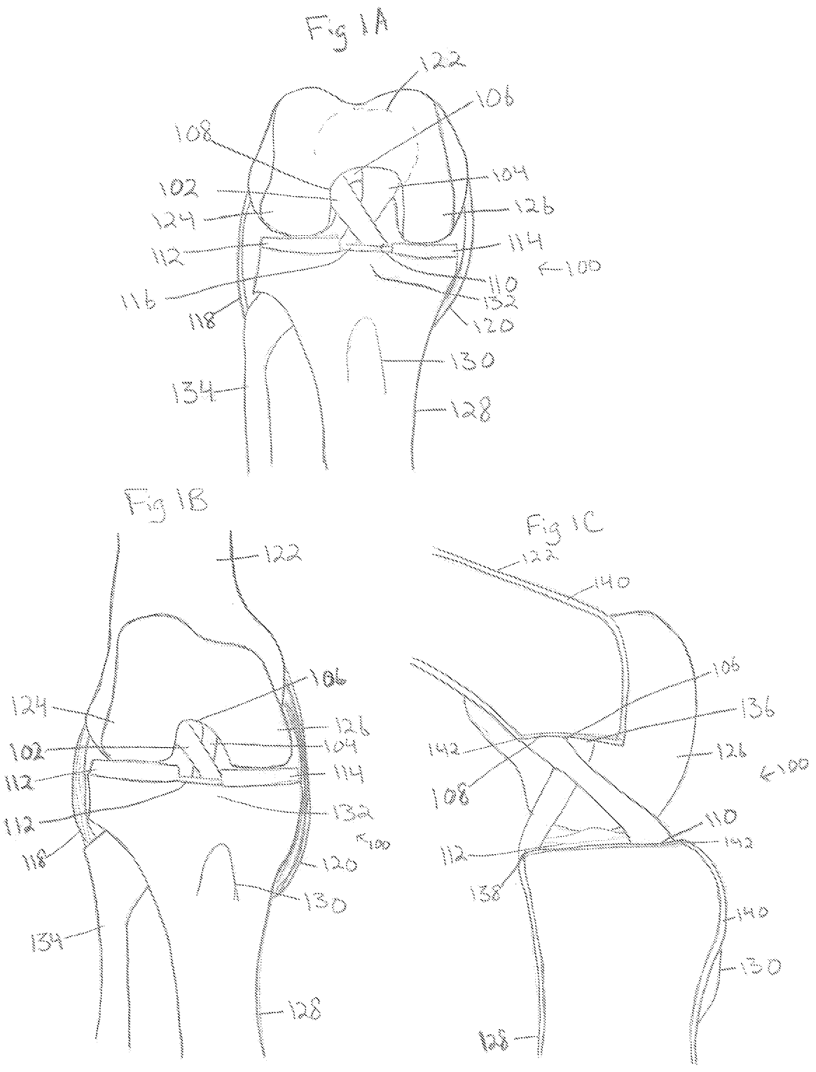

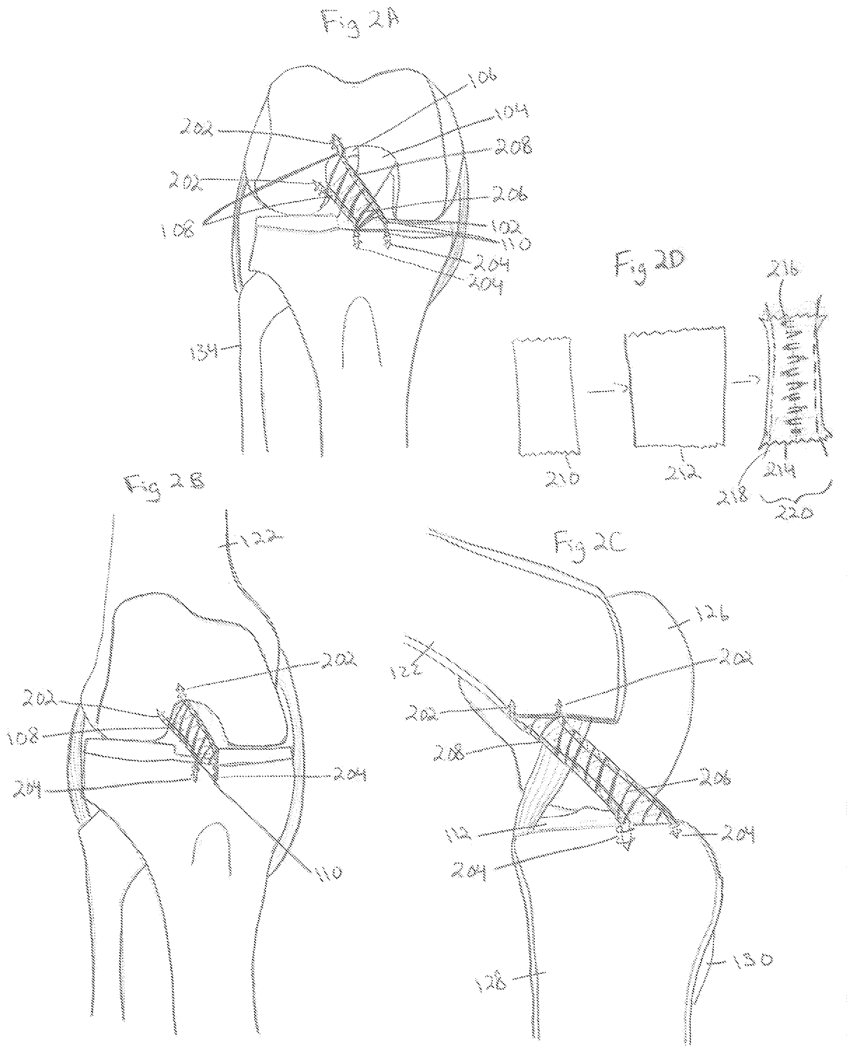

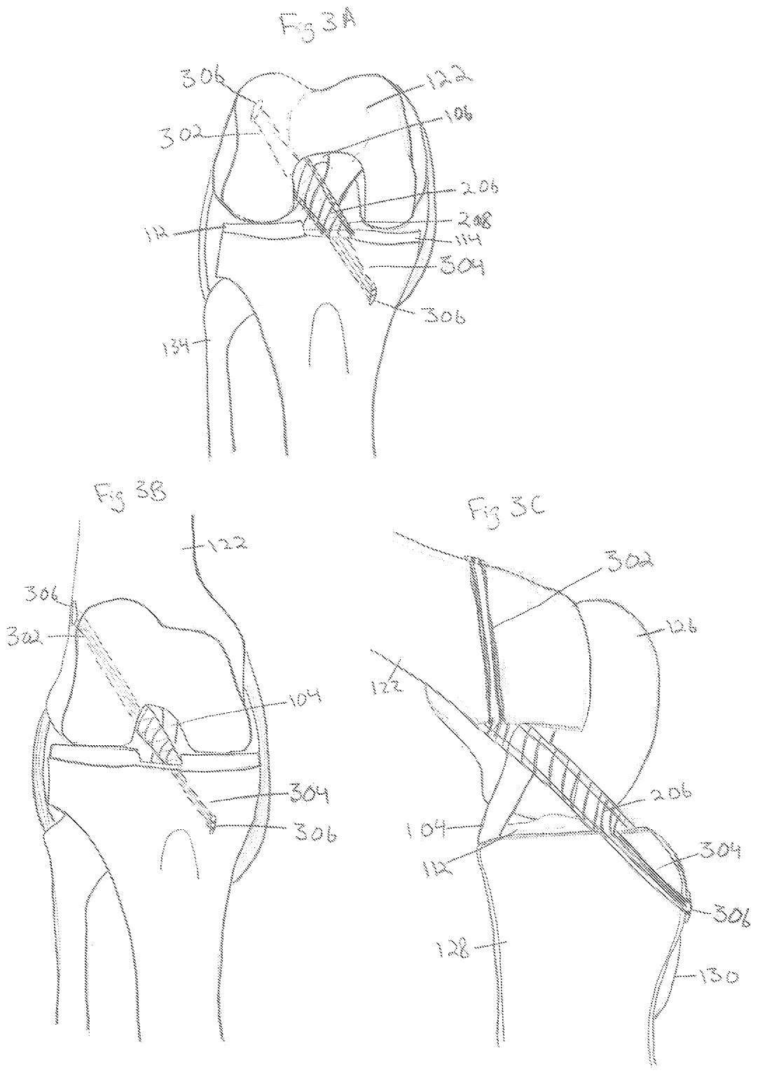

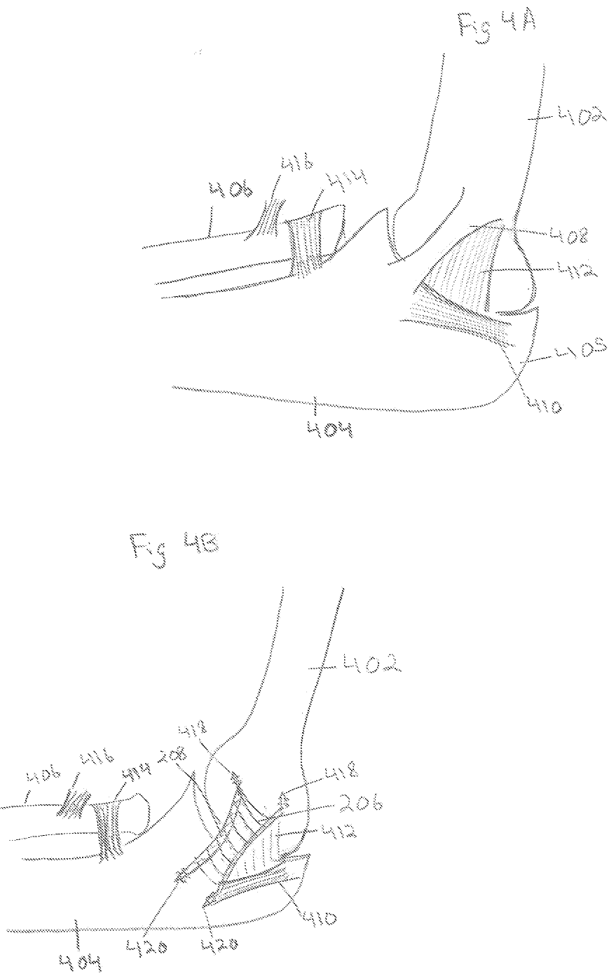

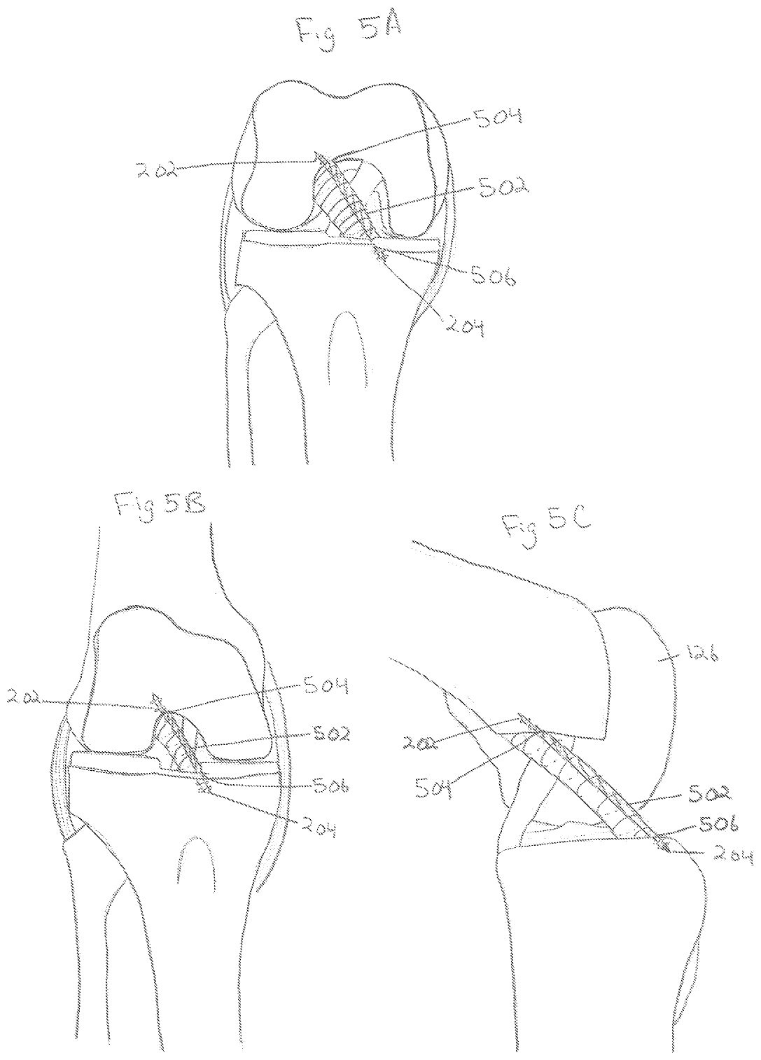

### CONCLUSION, RAMIFICATIONS, AND SCOPE

Thus, the reader will see that the methods described in this invention provides supplementation to a native ligament that may decrease the risk of injury to said ligament in the future. The complex created by the bundling of the graft with the native ligament and fixation to surrounding tissues and/or bone increases the strength and capacity of the complex to undergo stress, thus making the subject less likely to experience significant injury that may otherwise have required surgery or prolonged recovery.

While the above description and illustrations contain many specificities, these should not be construed as limitations on the scope of the invention, but rather as an exemplification of an embodiment thereof. Many other variations are possible as indicated in the alternative embodiments.

Accordingly, the scope of the invention should be determined not by the embodiments illustrated, but by the detailed descriptions, comprehensive list of embodiments, as well as the appended claims and their legal equivalents.

What is claimed is:

1. A method for supplementing a native ligament in a human, animal, or other biological subject, the method comprising: identification of elevated risk for ligament injury, creation of a graft-native ligament complex apparatus, attachment of said apparatus to bone and/or native ligament attachment site, achievement of a ligament complex that is more resistant to injury than previous.

2. The method of claim 1 wherein the identification of elevated risk for ligament injury comprises clinical, radiographic, biologic, physiologic, hormonal, genetic, chemical, mechanical, psychologic, technical, and/or activity related factors.

3. The method of claim 1 wherein the graft-native ligament complex comprises an allograft and native ligament.

4. The method of claim 1 wherein the graft-native ligament complex comprises an autograft and native ligament.

5. The method of claim 1 wherein the graft-native ligament complex comprises a synthetic graft and native ligament.

6. The method of claim 1 wherein the apparatus fixation is achieved by one or more suture anchors.

7. The method of claim 1 wherein the apparatus fixation is achieved by one or more cortical fixation devices utilizing one or more bone bridges/tunnels.

8. The method of claim 1 wherein the apparatus fixation is achieved by a biologic or synthetic adhesion material.

9. The method of claim 1 wherein the apparatus fixation is achieved by suture, staple, or other material used for the purpose of achieving stability of apparatus to native tissue.

10. The method of claim 1 wherein the creation of said apparatus is achieved with the aid of local tissue damage in order to promote healing.

11. The method of claim 1 wherein the creation of said apparatus is achieved with the aid of sutures, staples, patches, or other form of adhesion material.

12. The method of claim 1 wherein robotic assistance is utilized to predict, perform, analyze and/or evaluate the method.

13. The method of claim 1 wherein machine learning is utilized to predict, perform, analyze and/or evaluate the method of intervention.

14. An apparatus for supplementing a native, uninjured ligament in a human, animal, or other biological subject, consisting of a graft and said native ligament.

15. The apparatus of claim 14 wherein the apparatus consists of a graft attached and circumferentially wrapped around said native ligament.

16. The apparatus of claim 14 wherein the apparatus consists of a graft attached adjacent to said native ligament.

17. The apparatus of claim 14 wherein the apparatus consists of an allograft and said native ligament.

18. The apparatus of claim 14 wherein the apparatus consists of an autograft and said native ligament.

19. The apparatus of claim 14 wherein the apparatus consists of a synthetic graft or synthetic ingrowth material and said native ligament.

20. The apparatus of claim 14 wherein the apparatus consists of a graft unattached to the native ligament but in a position thought to decrease the tension on the native ligament and/or decrease risk of injury to said ligament.
